(19) United States
(12) Patent Application Publication
(10) Pub. No.: US 2005/0113666 A1
(43) Pub. Date: May 26, 2005

(54) Electroencephalograph sensor for use with magnetic resonance imaging and methods using such arrangements

(75) Inventors: Giorgio Bonmassar, Lexington, MA (US); John R Ives, Manotick (CA); John W Belliveau, Boston, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 10/505,577

(22) PCT Filed: Feb. 25, 2003
(86) PCT No.: PCT/US03/05614

Related U.S. Application Data
(60) Provisional application No. 60/360,203, filed on Feb. 28, 2002.

(51) Int. Cl. A61B 5/04

(52) U.S. Cl. 600/410; 600/544

(57) ABSTRACT

A signal recording system and a method for recording such signals is provided. In particular, a device is operable to be removably attached to a subject and to obtain signals (e.g., electroencephalogram (“EEG”) signals) from the subject. The device includes an amplifier and an electrode or a sensor. For example, the amplifier cna provide a radion frequency attenuation to the subject, and the amplifier can be mounted on the electrode, provided within the electrode, provided in the vicinity of the electrode, etc.

### CROSS-REFERENCE TO RELATED APPLICATION

This application claims priority from U.S. Provisional Patent Application No. 60/360,203 entitled “Circuit Arrangement Which Includes an Active Amplifier Incorporated Therein, and Methods for Utilizing Such Circuit Arrangement,” the disclosure of which is incorporated herein by reference in its entirety.

### FIELD OF THE INVENTION

The present invention relates generally to a signal recording arrangement and a method for recording such signals. In particular, the present invention is directed to a signal recording arrangement and a method for recording such signals in which a device for recording such signals from a subject includes an amplifier and an electrode or a sensor.

### BACKGROUND OF THE INVENTION

An electroencephalogram (“EEG”) machine is used to record the electrical activity in the brain of a patient. The procedure is performed by attaching a multitude of electrodes to the patient's scalp, and amplifying the recorded electrical signals. Magnetic resonance imaging (“MRI”) is a technique which utilizes magnetic and radio frequency (“RF”) fields to elicit a response from the tissue and provide high quality images of the inside of the human body along with detailed metabolic and anatomical information. During the 1990's, doctors recognized that the simultaneous recording of the EEG data and the MRI data can provide certain benefits which could not previously be realized with either method alone.

However, the combination of these two technologies poses several problems. The first is one of the measurement integrity. The changing magnetic and radio frequency (“RF”) fields can introduce undesirable artifacts into the EEG recordings. Moreover, the presence of ferromagnetic material within the EEG electrodes within the bore of the MRI apparatus and the electromagnetic radiation emitted by the EEG equipment can compromise the quality of the MRI image.

Several measures have been previously used to alleviate these measurement problems associated with the concurrent use of the EEG equipment and the MRI techniques. One such possibility is to replace the conventional electrodes with the electrodes composed of non-ferromagnetic materials, such as carbon fiber. Another possibility is to rearrange the EEG equipment leads which connect the electrodes to the EEG recording equipment. The placement and alignment of the EEG equipment leads within the MRI machine can have a substantial impact on the resultant image quality. This is because the EEG leads can interfere with the RF field by de-tuning the coils used in the magnetic resonance imaging, thereby resulting in a global attenuation of the received RF signal. U.S. Pat. No. 5,445,162 discloses a system which relocates the EEG recording equipment to a remote and isolated location that is external to the MRI room so as to minimize interference between the two systems. While these measures have assisted in improving the quality of both the EEG and MRI test results, certain problems still exist with such conventional systems and methods.

For example, the introduction of the EEG equipment into the pulsed RF fields (which are used to elicit MRI signals from the tissue of the subject) created by the MRI equipment presents a safety hazard, especially at high B0 fields because of the Specific Absorption Rate (“SAR”) considerations and the risk of burns. The pulsed or time-varying gradients and RF fields combined with the low impedance conduction of the subject (e.g., the patient) can induce current loops within the leads. While these loops normally have a high impedance due to the EEG amplifiers, various conditions can occur to provide a low impedance path, such as two leads coming into direct contact, a lead coming into direct contact with the patient, etc. These current loops can produce unsafe heat conditions, and may cause localized burns at the electrode contact points.

To alleviate these problems, it has been suggested to include the resistors in series with the electrodes to maintain a high impedance. (See K. Krakow et al., “Imaging of Interictal Epileptiform Discharges using Spike-Triggered fMRI”, I.J.B.E.M., 1 (1999); and R. Leahy et al., “A Study of Dipole Localization Accuracy for MEG and EEG Using a Human Skull Phantom”, Electroenchephalogy and Clin. Neurophysiol, 107 (1998), pp. 159-713). Certain materials, such as pure silver, silver-silver chloride and gold-coated silver electrodes, have also been implemented because they are non-magnetic, and therefore can be used safely. A carbon-fiber material is an advantageous material for the EEG leads since such material likely reduces the electromagnetic (“EM”) interference. Conductive plastic electrodes have also been employed, and may lower the amount of the EM interference and ballistocardiogram artifact. (See K. Krakow et al., “EEG-Triggered Functional MRI of Intertictal Epileptiform Activity in Patients with Partial Seizures”, Brain, 122 (1999), pp. 1679-1688).

In the absence of a ferromagnetic object implanted inside the human body, there is no replicated scientific study showing a health hazard associated with static magnetic field exposure, and there is likely no evidence of any hazards associated with the cumulative exposure to these magnetic fields. (See M. Schneider et al., “Magnetic Resonance Imaging—a Useful Tool for Airway Assessment”, Acta Anaesthesiol Scand, 33 (1989), pp. 429-431). However time-varying gradient magnetic fields (dB/dt) may stimulate nerves or muscles of the subject by inducing the electric fields in the subject. (See D. J. Schaefer, “Dosimetry and Effects of MR Exposure to RF and Switched Magnetic Fields”, Annals of the New York Academy of Sciences, 649 (1992), pp. 225). RF is probably of the most concern because during the magnetic resonance procedures, a significant amount of the RF transmitted power is transformed into heat in the patient tissue due to its resistivity. (See R. Weisskoff et al., “Microscopic Susceptibility Variation and Transverse Relaxation: Theory and Experiments”, Magn. Reson. Med., 31. (1994), pp. 601-610.) The visualization and quantification of RF heating of a tissue phantom during the MRI procedure is a safety procedure which allows an examination of the heating patterns of transmit/receive surface coils. (See S. Warach et al., “Hyperperfusion of Ictal Seizure Focus Demonstrated by MR Perfusion Imaging, AJNR, 15 (1994), pp. 965-968).

It was also proposed to directly connect the subject's leads to a junction box, and then extend them to a pre-amplifier (e.g., directly into a pre-set and hard-wired pre-amplifier). (See L. Lemieux et al., “Recording of EEG during fMRI Experiments: Patient Safety”, Magn. Reson. Med., 38 (1997), pp. 943-952; and K. K. Kwong et al., “Dynamic Magnetic Resonance Imaging of Human Brain Activity during Primary Sensory Stimulation”, Proc. Natl. Acad. Sci. USA, 89 (1992), pp. 5675-5679). However, the wire connections of such arrangement should be maintained as short as possible in order to keep the noise levels within tolerable limits. The pre-amplifier is then connected to the outside world through an isolated fiber-optic cable. Once outside, the signals can be fed into a Biopotential Amplifier, an EEG system or a variety of data acquisition devices. (See R. Price, “The AAPM/RSNA Physics Tutorial for Residents. MR Imaging Safety Considerations”, Radiological Society of North America., Radiographics, 19 (1999), pp. 1641-51; and P. T. Fox et al., “Nonoxidative Glucose Consumption during Focal Physiologic Neural Activity”, Science, 241 (1988), pp. 462-464.)

While alleviating the above-described problems to some extent, there still exists a need for a safer method and circuit arrangement for introducing the EEG equipment into the bore of the MRI device.

### SUMMARY OF THE INVENTION

The present invention is directed to a signal recording system and a method for recording such signals (I, for recording electroencephalogram (“EEG”) signals during a magnetic resonance imaging (“MRI”) procedure). More specifically, a device for recording such signals from a subject includes an amplifier and an electrode or a sensor to allow an EEG recording to be performed during the MRI procedure with a higher degree of subject safety and lower noise levels.

The combination of the EEG and MRI recordings provides benefits to a neuroscientist that may be not achieved with either method alone. However, one exemplary shortcoming of the prior art methods and apparatuses is that the pulsed radio frequency (“RF”) fields which are used to elicit the MRI results from subject's tissue may provoke heating thereof in closed current loops of the EEG electrodes at the electrode contact points. Such heating can possibly result in a bodily injury to the subject ( burns, electric shock, etc.). This problem is exacerbated by the fact that these disadvantageous current loops are not usually detected by the subject since the sensory perception is dominated at these relatively high frequencies by a thermal sensitivity.

The present invention also relates to an active EEG electrode system with a built-in insthunentation amplifier circuit mounted on the electrodes which provides an RF attenuation to the subject, thus alleviating some of the risks associated with the induced current loops. As an additional benefit, the above-described arrangement of active electrodes can improve the signal quality of the EEG results. In another embodiment of the present invention, the active electrode arrangement has the ability to subtract the artifact noise directly from the raw EEG signal. This can be performed by utilizing the FPAA technology (or programmable analog circuits) such as the ispPAC30 (Max Dim 10 mm×10 mm) Lattice Semiconductor, Hillsboro, Oreg.

These and other advantages can be realized with a first exemplary embodiment of the present invention, in which a signal recording arrangement and method for recording such signals are provided, such that a device operable to be removably attached to a subject and to obtain signals (e.g., electroencephalogram (“EEG”) signals) from the subject includes an amplifier and an electrode or a sensor. For example, the amplifier can provide a radio frequency attenuation to the subject, and the amplifier can be mounted on the electrode, provided within the electrode, provided in the vicinity of the electrode, etc. The amplifier may include an amplifier circuit, and the amplifier circuit may include a resistor (e.g., a 10,000 Ohm resistor). Moreover, the device can be provided within a magnetic resonance imaging (“MRI”) environment.

The arrangement may also include a processing system coupled to the device. Specifically, the processing system can be operable to receive the EEG signals, to process the EEG signals so as to generate EEG data, and to display the EEG data to a user of the processing system. The processing system may be arranged externally from the MRI environment. The arrangement can also comprise a transmitter (e.g., an optilink system transmitter) coupled to the device, and a receiver (e.g., an optilink system receiver) coupled to each of the transmitter and the processing system. Specifically, the transmitter may be operable to receive the EEG signals from the device and to transmit the EEG signals to the receiver via an optical fiber arrangement, and the receiver may be operable to receive the EEG signals from the transmitter and to transmit the EEG signals to the processing system. Moreover, the transmitter can be provided within the MRI environment, and the receiver can be arranged externally from the MRI environment.

In a modification of the first exemplary embodiment of the present invention, after the device obtains the EEG signals from the subject, the device may filter one or more artifact noise signals (e.g., radio frequency field signal, a magnetic field signal, etc.) from the EEG signals so as to generate filtered EEG signals. In this embodiment, the transmitter may be operable to receive the filtered EEG signals from the device and to transmit the filtered EEG signals to the receiver via the optical fiber arrangement. The receiver may be operable to receive the filtered EEG signals from the transmitter and to transmit the filtered EEG signals to the processing system. Moreover, the processing system may be operable to receive the filtered EEG signals from the receiver and to process the filtered EEG signals so as to generate filtered EEG data.

In another modification of the first exemplary embodiment of the present invention, the receiver can include a demultiplexer which may be operable to demultiplex the EEG signals so as to generate demultiplexed EEG signals. In this embodiment, the processing system can be replaced by an EEG system coupled to the receiver. The EEG system may be operable to receive the demultiplexed EEG signals from the receiver and to process the demultiplexed EEG signals so as to generate demultiplexed EEG data.

In a second exemplary embodiment of the present invention, the device is operable to obtain the signals (e.g., the EEG signals) from the subject, and includes the amplifier and the electrode or the sensor. Moreover, an EEG system may be coupled to the device, which is operable to receive the EEG signals from the device. For example, each of the EEG system and the device can be positioned within the MRI environment, and the EEG system may generate MRI signals based on the EEG signals. In the second exemplary embodiment, a processing system can be coupled to the EEG system by an optical link, and the processing system can be operable to receive the EEG signals and the MRI signals from the EEG system. The processing system can be arranged externally from the MRI environment. Moreover, the processing system may be operable to process the EEG signals so as to generate EEG data, and to process the MRI signals so as to generate MRI data.

Unless otherwise defined, all technical and scientific terms used herein have the same, or substantially similar, meaning as commonly understood by one of ordinary skill in the art to which the present invention belongs. Although processes, methods and systems similar or equivalent to those described herein can be used in the practice or testing of the present invention, exemplary processes, systems and software arrangements are described below in further detail. In addition, the systems, processes, and examples are provided for the purposes of illustration only, and are in no way limiting. All cited references are incorporated herein by reference.

Other objects, features, and advantages will be apparent to persons of ordinary skill in the art from the following detailed description of the invention and the accompanying drawings.

### BRIEF DESCRIPTION OF THE DRAWINGS

For a more complete understanding of the present invention, the needs satisfied thereby, and the objects, features, and advantages thereof, reference now is made to the following description taken in connection with the accompanying drawings.

FIG. 1 is a high level block diagram of a first exemplary embodiment of a magnetic resonance imaging (“MRI”) system according to the present invention which utilizes. the active amplifier incorporated with an electrode arrangement that is coupled to a processing system.

FIG. 2 is a high level block diagram of a second exemplary embodiment of the MRI system according to the present invention which utilizes the active amplifier incorporated with the electrode arrangement that is coupled to an electroencephalogram (“EEG”) system.

FIG. 3 is a high level block diagram of a third exemplary embodiment of the MRI system according to the present invention which utilizes the active amplifier incorporated with the electrode arrangement that does not use an optilink-type communication arrangement.

FIG. 4 is a visual illustration of an exemplary embodiment of the electrode shown in FIGS. 1-3, which incorporates therewith the active amplifier.

FIG. 5 is a high level diagram of an exemplary embodiment of the active amplifier shown in FIGS. 1-3.

FIG. 6 is a detailed schematic diagram of the active amplifier shown in FIG. 5.

FIG. 7 is a graph comparing EEG traces recorded over a particular period of time using a conventional EEG electrode set and an exemplary embodiment of an active EEG electrode set according to the present invention.

### DETAILED DESCRIPTION

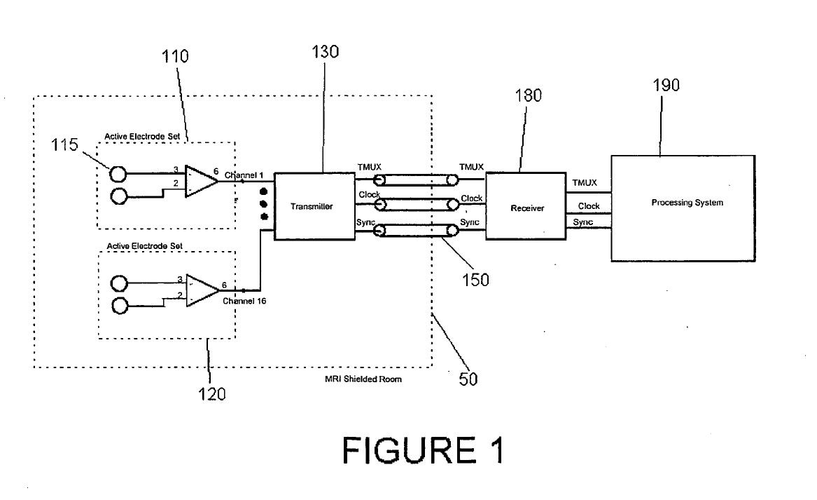

Preferred embodiments of the present invention and their features and advantages may be understood by referring to FIGS. 1-7, like numerals being used for like corresponding parts in the various drawings.

Combining evoked potential recordings with the fMRI equipment can provide a neuroscientist with a higher spatiotemporal resolution than either method alone. By concurrently recording EEG or ERP with the fMRI equipment, it is possible to establish that these measurements reflect the same brain activity state which provides an accurate characterization of the location and timing of a neuropsychological activity in the human brain. Furthermore, clinical applications of this technology are becoming more common, especially in epilepsy research. (See E. R. Alexander et al., “The Present and Future Role of Intraoperative MRI in Neurosurgical Procedures”, Stereotact Funct. Neurosurg., 68 (1997), pp. 10-17; and P. J. Allen et al., “Identification of EEG Events in the MR Scanner: the Problem of Pulse Artifact and a Method for its Subtraction”, Neuroimage, Vol. 8 (1998), pp. 229-239). However, the conventional EEG techniques and monitoring equipment may interfere with the technical demands of the MRI techniques. The pulsed radio frequency fields, which are used to elicit MRI signals from tissue, may provoke heating in closed loops of EEG/ECG electrodes and cause bodily injuries to the subject. These injected currents are usually not detected by the subject since the sensory perception is dominated at these relatively high frequencies by a thermal sensitivity. The Specific Absorption Rate (“SAR”) may also rise due to the presence of the EEG leads that may act as antennas.

Referring to FIG. 1, a high level block diagram of a first exemplary embodiment of a magnetic resonance imaging (“MRI”) system according to the present invention which utilizes an active amplifier incorporated with a first electrode arrangement 110 that is coupled to a processing system 190, is shown. The electrode arrangement 110 can be an active electrode set which includes a pair of electrodes 115, such as a pair of wet scalp electrodes, a pair of dry surface electrodes, etc. The electrodes may be connected to a subject's body part to establish the subject's EEG measurements via that particular body part. It will be readily understood by those of ordinary skill in the art that one or more second electrode arrangements 120 can also be attached to the subject so as to obtain EEG and/or other types of measurements. Moreover, a guard or a shield (not shown) may be used to prevent RF signals from reaching circuitry of the active amplifier. (See C. J. Harland et al., “Remote Detection of Human EEG Using Ultrahigh Input Impedence Electric Potential Sensors”, Applied Physics Letters, Volume 81, Number 17 (Oct. 21, 2002).

Each of the electrode arrangements 110, 120 may be connected to an optilink system transmitter 130 (e.g., an optilink system transmitter manufactured by Neuroscan, El Paso, Tex.), via a separate respective channel. The optilink system transmitter 130 transmits the subject's measurements to a remote optilink system receiver 180 via optical fibers of an optical fiber arrangement 150 which carry the signals (e.g., 16-bipolar channels time multiplexed signals) from the transmitter 130 to the receiver 180. The electrode arrangements 110, 120 and the optilink system transmitter 130 can be provided in an MRI shielded room 50 which prevents the pulsed radio frequency fields from disrupting the operation of the devices arranged externally from this room 50. In an exemplary embodiment of the present invention, only the fibers of the optical fiber arrangement 150 carry the data from the transmitter 130 to the receiver 180. In another exemplary embodiment of the present invention, a separate fiber (or set of fibers) of the optical fiber arrangement 150 can carry time-multiplexed signals, clock signals and synchronization signals, respectively. The optilink system receiver 180 then may forward the data received from the optilink system transmitter 130 to the processing system 190, which can be a personal computer (e.g., a laptop personal computer) which has a DAQCard 16xx card (E.g., a 16 Bit A/D PCMCIA card) that processes the data, and is operable to output the results of the analysis (and/or the readings) on a display or printer device. This information can also be forwarded to other one or more processing systems for further analysis.

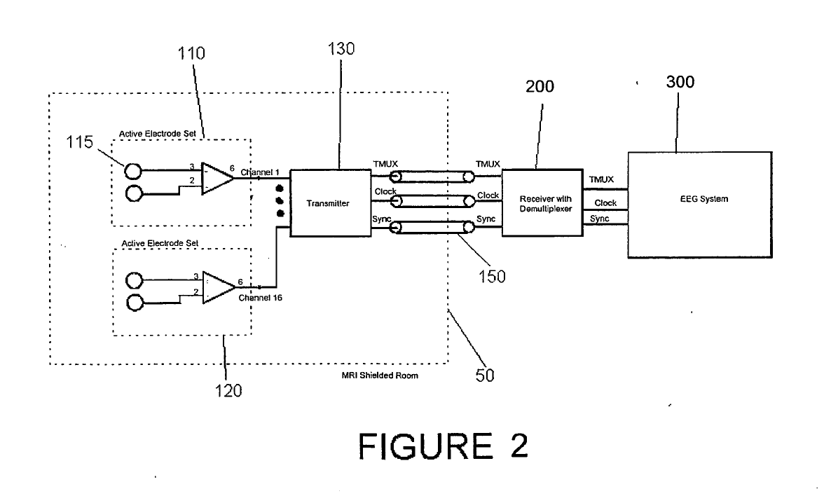

Referring to FIG. 2, a high level block diagram of a second exemplary embodiment of the MRI system which utilizes the active amplifier incorporated with an electrode arrangement, is shown. In this exemplary embodiment of the present invention, an optilink system receiver 200 can be coupled to an electroencephalogram (“EEG”) system 300. The optilink system receiver 200 may include a demultiplexer which can be used to demultiplex the signals received from the optilink system transmitter 130, and forward separate demultiplexed data to the convention EEG system 300 via a particular number of channels. In an exemplary embodiment of the present invention, the number of channels that are used to provide the data from the optilink system receiver 200 can be equal to the number of the channels utilized for transmitting data from the electrode arrangements 110, 120 to the optilink system transmitter 130 (e.g., 34 channels).

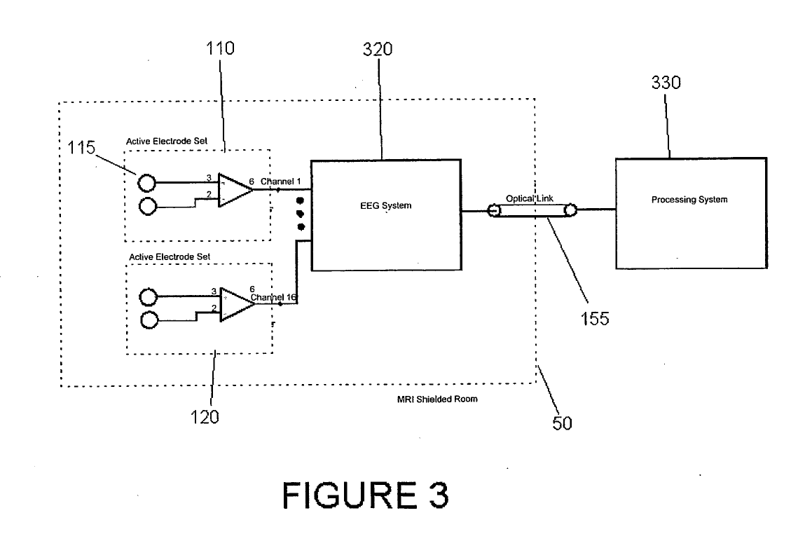

Referring to FIG. 3, a high level block diagram of a third exemplary embodiment of the MRI system which utilizes the active amplifier incorporated with the electrode arrangements that does not use an optilink-type communication, is shown. In particular, each of the electrode arrangements 110, 120 can be connected to (e.g., directly connected to ) another EEG system 320 which can also be provided in the MRI shielded room 50. This EEG system 320 may utilize the data received from the electrode arrangements 110, 120 to obtain MRI measurements, and can receive the data from the electrode arrangements 110, 120 via, e.g., 32 channels. The EEG system 320 can then forward the MRI measurements outside of the MRI shielded room 50 to a personal computer 330 via an optical link 155. An example of such EEG system 320 can be an “ActiveOne” system, sold by Cortech Solutions L.L.C., Wilmington, N.C.

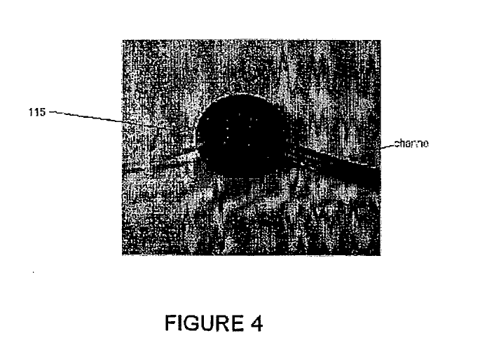

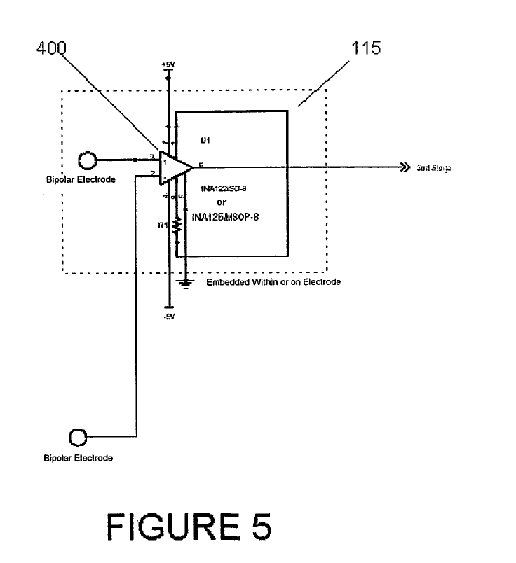

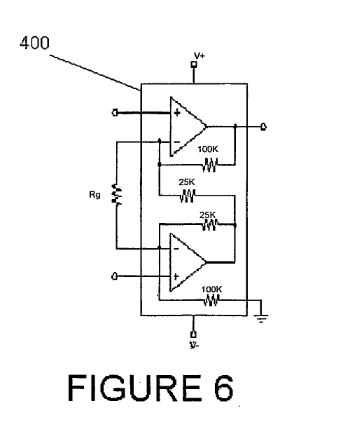

Referring to FIG. 4, a visual illustration of an exemplary embodiment of one of the electrodes of the electrode arrangements 110, 120 shown in FIGS. 1-3, which incorporates therewith (or surface mounts thereon) the active amplifier so as to provide an RF attenuation to the subject, is shown. The electrodes of the electrode arrangements 110, 120 can be plastic-conductive electrodes (as described in Bonmassar G. et al. “Visual Evoked Potential (VEP) Measured by Simultaneous 64-Channel EEG and 3T FMRI”, NeuroReport. 10, 1999, pp. 1893-1897) which can be coated with silver epoxy that is made by Chemtronics of Kemesaw, Ga. These electrodes can be electrically bonded to a conductive fiber (8.5Σ/in±12%—Fiberohm, Marktek Inc., Chesterfield, Mich.) using, e.g., a silver epoxy. The electrodes 115 can be placed on the skin of the subject using an EEG paste (e.g., Elefix, Nihon Kohden, Tokyo, Japan). It is also possible to use silicone (P.N. 25827, Loctite Corp., Rocky Hill, Conn.) to provide a mechanical stability for holding together all components, and preventing tearing of the cables. The amplifier used with the electrode arrangements 110, 120 according to the present invention can be Burr-Brown INA122 amplifier and/or Burr-Brown INA126 amplifier, which can be utilized as an instrumentation amplifier. This exemplary amplifier 400 is illustrated in FIG. 5 with external wires for +5V, −5V, ground and output. Additional details of the Burr-Brown INA122 and INA126 amplifiers are provided in the specifications of the Burr-Brown INA122 and INA126 amplifier, the disclosure of which being incorporated herein by reference. FIG. 6 shows a detailed schematic diagram of the active amplifier 400 illustrated in FIG. 5. In this diagram, a resistor R_{G} of the amplifier can be selected to be about 10K Ohms, such as to achieve a G value of about 25, in which G=5 +(200K/R_{G}).

According to another exemplary embodiment of the present invention, the measurements of the noise by the electrode arrangements 110, 120 allow for a direct amplification therefrom, and possibly from a set of conventional EEG electrodes. The conventional electrodes set may have non-metallic FiberOhm leads. Moreover, the electrodes can be composed of a conductive plastic material with a thin layer of silver epoxy coating. The signals obtained by the electrodes can be A/D converted at 24-bit rate directly inside the fringe field with a sampling frequency, e.g., up to 1,000 S/s. These converted signals can also be post-processed by MATLAB® software using a band-pass Chebyshev Type I IIR filter of order 8 (lowpass) and 5 (highpass) with a band between 0.1 Hz to 70 Hz.

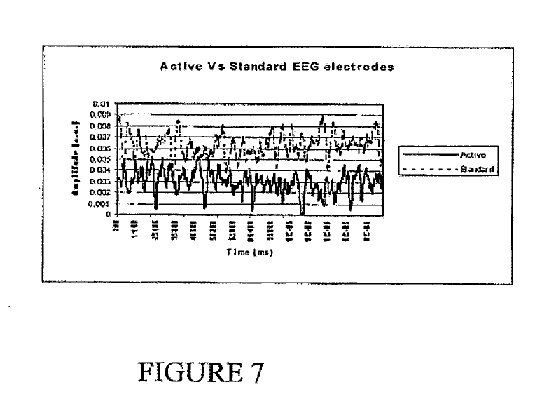

Referring to FIG. 7, a graph of measurements versus time performed on two subjects during a rest condition to measure ballistocardiogram noise therefor, is shown. In this graph, the EEG traces exhibit a lower peak-to-peak noise when collected from the active electrode pair 115 of the present invention (−66:V to +96:V) compared to those of the conventional electrode pair (−190:V to 86:V). The variance is also estimated for these recordings and the active electrodes exhibited a lower signal variance (6.2 10⁻¹²V²) compared to the use of the conventional electrodes (8.1 10⁻¹²V²). The above values provide a clear indication that the active electrodes are capable of better signal to noise (“SNR”) recordings. This is because at this field strength, a significant amount of the variance is due to the ballistocardiogram noise. Thereafter, the MRI procedure can be performed to analyze the effect of the active EEG electrodes inside the head coil and the possible presence of artifacts. The exemplary images provide a sufficient quality, and are thus beneficial and usable for TRI studies. The observed signal drop in correspondence to electrode location can be similar for active and passive electrodes.

In yet another embodiment of the present invention, an adaptive filtering technique can be implemented with the electrode arrangements 110, 120. The active electrodes 115 may be operable to subtract the artifact noise directly from the raw EEG signal. It is possible to utilize an FPAA technology (or programmable analog circuits) such as the ispPAC30 (Max Dim 10 mm×10 mm) Lattice Semiconductor, Hillsboro, Oreg. (the details of which are provided in the specification thereof, the entire disclosure being incorporated herein by reference). A differential signaling type of circuit can be utilized which may be similar to the adaptive filter, without the time-variant component. The details of this technique are set forth in the manuscript by Giorgio Bonmassar et al., “Motion and Ballistocardiogram Artifact Removal for Interleaved Recording of EEG and EPs during MRI”, NMR Center, Massachusetts General Hospital, Harvard Medical School, and A. Martinos Center for Biomedical Imaging.

While the invention has been described in connection with preferred embodiments, it will be understood by those skilled in the art that other variations and modifications of the preferred embodiments described above may be made without departing from the scope of the invention. Other embodiments will be apparent to those skilled in the art from a consideration of the specification or practice of the invention disclosed herein. It is intended that the specification and the described examples are consider exemplary only with the true scope of the invention indicated by the following claims.

1. A signal recording arrangement, comprising:
at least one device operable to be removably attached to at least a portion of a subject, and to obtain at least one signal from the subject, wherein the at least one device includes at least one of an electrode and a sensor, and comprises an amplifier.

2. The arrangement of claim 1, wherein the amplifier is operable to provide a radio frequency attenuation to the subject.

3. The arrangement of claim 2, wherein the amplifier comprises an amplifier circuit, and wherein the amplifier circuit comprises at least one resistor.

4. The arrangement of claim 3, wherein a resistance of the at least one resistor is about 10,000 Ohms.

5. The arrangement of claim 1, wherein the at least one device is provided within a magnetic resonance imaging environment.

6. The arrangement of claim 1, wherein the amplifier is one of mounted on the electrode, provided within the electrode, and provided in the vicinity of the electrode.

7. The arrangement of claim 1, wherein the at least one signal comprises at least one electroencephalogram (“EEG”) signal.

8. The arrangement of claim 7, further comprising a processing system coupled to the at least one device, wherein the processing system is operable to receive the at least one EEG signal, and to process the at least one EEG signal so as to generate EEG data.

9. The arrangement of claim 8, wherein the processing system is further operable to display the EEG data to a user of the processing system.

10. The arrangement of claim 8, wherein the at least one device is provided within a magnetic resonance imaging (“MRI”) environment, and wherein the processing system is arranged externally from the MRI environment.

11. The arrangement of claim 8, further comprising:
a transmitter coupled to the at least one device, wherein the transmitter is operable to receive the at least one EEG signal from the at least one device, and to transmit the at least one EEG signal via an optical fiber arrangement; and
a receiver coupled to each of the transmitter and the processing system, wherein the receiver is operable to receive the at least one EEG signal from the transmitter via the optical fiber arrangement, and to transmit the at least one EEG signal to the processing system.

12. The arrangement of claim 11, wherein the transmitter comprises an optilink system transmitter, and wherein the receiver comprises an optilink system receiver.

13. The arrangement of claim 11, wherein each of the at least one device and the transmitter is provided within a magnetic resonance imaging (“MRI”) environment, and wherein each of the receiver and the processing system is arranged externally from the MRI environment.

14. The arrangement of claim 7, wherein the at least one device is further operable to filter at least one artifact noise signal from the at least one EEG signal so as to generate at least one filtered EEG signal.

15. The arrangement of claim 14, wherein the at least one artifact noise is associated with at least one of a radio frequency field and a magnetic field.

16. The arrangement of claim 14, further comprising:
a transmitter coupled to the at least one device, wherein the transmitter is operable to receive the at least one filtered EEG signal from the at least one device, and to transmit the at least one filtered EEG signal via an optical fiber arrangement;
a receiver coupled to each of the transmitter and the processing system, wherein the receiver is operable to receive the at least one filtered EEG signal from the transmitter via the optical fiber arrangement, and to transmit the at least one filtered EEG signal; and
a processing system coupled to the receiver, wherein the processing system is operable to receive the at least one filtered EEG signal from the receiver, and to process the at least one filtered EEG signal so as to generate EEG data.

17. The arrangement of claim 16, wherein each of the at least one device and the transmitter is provided within a magnetic resonance imaging (“MRI”) environment, and wherein each of the receiver and the processing system is arranged externally from the MRI environment.

18. The arrangement of claim 7, further comprising an EEG system coupled to the at least one device, wherein the EEG system is operable to receive the at least one EEG signal from the at least one device.

19. The arrangement of claim 18, wherein each of the EEG system and the at least one device is positioned within a magnetic resonance imaging (“MRI”) environment.

20. The arrangement of claim 19, wherein the EEG system is further operable to generate at least one MRI signal based on the at least one EEG signal.

21. The arrangement of claim 20, further comprising a processing system coupled to the EEG system by an optical link, wherein the processing system is operable to receive the at least one EEG signal and the at least one MRI signal from the EEG system.

22. The arrangement of claim 21, wherein the processing system is further operable to process the at least one EEG signal so as to generate EEG data, and to process the at least one MRI signal so as to generate MRI data.

23. The arrangement of claim 22, wherein the processing system is arranged externally from the MRI environment.

24. The arrangement of claim 7, further comprising a receiver coupled to the at least one device, wherein the receiver is operable to receive the at least one EEG signal from the at least one device, wherein the receiver comprises a demultiplexer, and wherein the demultiplexer is operable to demultiplex the at least one EEG signal so as to generate at least one demultiplexed EEG signal.

25. The arrangement of claim 24, further comprising an EEG system coupled to the receiver, wherein the EEG system is operable to receive the at least one demultiplexed EEG signal from the receiver, and to process the at least one demultiplexed EEG signal so as to generate demultiplexed EEG data.

26. The arrangement of claim 25, further comprising a transmitter coupled to the at least one device, wherein the transmitter is operable to receive the at least one EEG signal from the at least one device, and to transmit the at least one EEG signal via an optical fiber arrangement to the receiver, wherein the receiver is operable to receive the at least one EEG signal from the transmitter via the optical fiber arrangement.

27. The arrangement of claim 26, wherein each of the at least one device and the transmitter is provided within a magnetic resonance imaging (“MRI”) environment, and wherein each of the receiver and the EEG system is arranged externally from the MRI environment.

28. A method for recording a signal, comprising the steps of:
obtaining at least one signal from a subject; and
providing a radio frequency attenuation to the subject.

29. The method of claim 28, wherein the obtaining step comprises the substep of obtaining the at least one signal within a magnetic resonance imaging environment.

30. The method of claim 29, wherein the at least one signal comprises at least one electroencephalogram (“EEG”) signal.

31. The method of claim 30, further comprising the step of processing the at least one EEG signal so as to generate EEG data.

32. The method of claim 31, further comprising the step displaying the EEG data.

33. The method of claim 30, further comprising the step of filtering at least one artifact noise signal from the at least one EEG signal so as to generate at least one filtered EEG signal.

34. The method of claim 33, wherein the at least one artifact noise is associated with at least one of a radio frequency field and a magnetic field.

35. The method of claim 34, further comprising the step of processing the at least one filtered EEG signal so as to generate EEG data.

36. The method of claim 30, further comprising the step of generating at least one MRI signal based on the at least one EEG signal.

37. The method of claim 36, further comprising the steps of:
processing the at least one EEG signal so as to generate EEG data; and
processing the at least one MRI signal so as to generate MRI data.

38. The method of claim 30, further comprising the step of demultiplexing the at least one EEG signal so as to generate at least one demultiplexed EEG signal.

39. The method of claim 38, further comprising the step of processing the at least one demultiplexed EEG signal so as to generate demultiplexed EEG data.
